(19) United States
(12) Patent Application Publication
(10) Pub. No.: US 2026/0284352 A1
(43) Pub. Date: Sep. 24, 2026

(54) System and method for delivering immersive therapeutic experiences for neurocognitive and sleep disorders through the use of virtual reality (vr) environments and synchronized 40hz sensory stimulation

(71) Applicants: Immanual Sundar Joseph, McKinney, TX (US); Jack Abbott, San Diego, CA (US)

(72) Inventors: Immanual Sundar Joseph, McKinney, TX (US); Jack Abbott, San Diego, CA (US)

(21) Appl. No.: 19/280,361

(22) Filed: Jul. 25, 2025

Related U.S. Application Data
(60) Provisional application No. 63/677,617, filed on Jul. 31, 2024.

(51) Int. Cl. A61M 21/02; A61M 21/00; A61N 5/06

(52) U.S. Cl. CPC A61M 21/02; A61N 5/0618; A61N 5/0622; A61M 2021/0027; A61M 2021/005; A61M 2205/507; A61N 2005/0647; A61N 2005/0663

(57) ABSTRACT

The present invention provides immersive therapeutic interventions to individuals with neurocognitive and sleep disorders using extended reality (XR) environments synchronized with approximately 40 Hz sensory stimulation. The system integrates synchronized 40 Hz light and sound stimulation with immersive, task-based XR engagement to support cognitive, functional, relaxation, and motor-rehabilitation activities in a non-invasive format. In some embodiments, the system logs session-related data, including task completion and engagement metrics, for caregiver or clinician review and longitudinal progress tracking.

### CROSS-REFERENCE TO RELATED APPLICATIONS

This application claims the benefit of U.S. Provisional Application No. 63/677,617, filed Jul. 31, 2024, titled “Virtual Reality Cognitive Therapy System With 40Hz Light And Sound Stimulation,” the entirety of which is incorporated herein by reference.

A system and method for delivering immersive therapeutic experiences for neurocognitive and sleep disorders through the use of extended reality (XR) environments and synchronized 40 Hz sensory stimulation

### FIELD OF THE INVENTION

The present invention relates generally to a system and method for cognitive support and neurorehabilitation. More specifically, it relates to systems and methods that synergistically combine virtual reality (VR) and extended reality (XR) environments with 40 Hz sensory stimulation for individuals with neurocognitive and sleep disorders.

### BACKGROUND

Cognitive impairments arising from neurological conditions such as stroke, traumatic brain injury (TBI), Alzheimer's disease, and other neurocognitive disorders can severely impact memory, attention, executive functioning, and daily living skills. Traditional therapies often lack real-world relevance and engaging content, limiting their effectiveness.

Extended Reality (XR), including Virtual Reality (VR), offers immersive, task-relevant simulations that improve motivation and ecological validity in therapy. Meanwhile, emerging studies indicate that 40-Hertz light and sound stimulation can enhance gamma brainwave activity, thereby promoting neuroplasticity and potentially reducing pathological markers associated with neurocognitive disorders.

Several patents have attempted to address brain stimulation and cognitive training using light, sound, or virtual environments.

US20190076670A1 discloses flashing light therapy combined with cognitive games for dementia care. However, it is screen-based, lacks audio stimulation, and is not immersive.

EP4354454A1 covers a VR system for neurological disease therapy, but does not focus on 40 Hz entrainment or bilateral motor engagement.

US20230218854A1 involves VR-based brainwave entrainment through gamified objects, but lacks therapeutic task design and functional goal orientation.

While these references suggest components of the broader concept, none disclose a unified system that integrates: synchronized 40 Hz visual and auditory stimulation; immersive XR-based experiences; structured simulations of functional tasks such as shopping, cooking, or dressing utilizing natural hand tracking instead of controllers; bilateral motor engagement for neurorehabilitation; and deployment for therapeutic goals across neurocognitive conditions and sleep disorders.

### SUMMARY OF THE INVENTION

An aspect of the present invention is to provide a comprehensive system and method to deliver immersive therapeutic interventions through a VR or XR platform synchronized with 40 Hz light and sound stimulation.

Another aspect of the invention is the use of commercially available XR or VR headsets to deliver multisensory, immersive 360-degree experiences.

A further aspect of the invention is visual stimulation through flickering light at either 480 nm or 660 nm wavelengths pulsed at approximately 40 Hz, supporting circadian alignment and neural health.

Another aspect of the invention is auditory stimulation at approximately 40 Hz using binaural or monaural beats, pulses, or rhythmic sound output phase-locked with the 40 Hz flickering light.

Still further aspects of the invention include interactive scenarios that support memory, attention, language, and executive function recovery.

Another aspect of the invention is bilateral engagement to support motor rehabilitation, including rehabilitation following stroke.

Another aspect of the invention includes content libraries covering functional domains such as Foundational Skills, Activities of Daily Living (ADLs), Instrumental Activities of Daily Living (IADLs), Social Participation, and Relaxation and Wellness. These activities may utilize natural hand tracking without the need for controllers and may be accessed offline.

A further aspect of the invention is that the system is designed for use in clinical, residential, or home settings, with therapy sessions initiated through a simplified user interface. Sessions include light and sound stimulation synchronized with VR or XR content. The system may log user sessions to support future analysis or longitudinal monitoring.

### BRIEF DESCRIPTION OF THE DRAWINGS

FIG. 1 illustrates an overview of the XR therapeutic system in which phase-locked 40 Hz visual and auditory stimulation and XR therapeutic content are delivered through commercially available VR headsets, with access managed through a mobile device management (MDM) or content management system and session logs used to inform subsequent sessions.

FIG. 2 illustrates a system architecture and content delivery workflow showing 40 Hz-reinforced XR content, MDM or content delivery management, VR headsets, simplified user access, and representative content categories including cognitive stimulation, relaxation and wellness, and living skills activities.

FIG. 3 illustrates an example user flow for a virtual shopping or living-skills simulation in which the user receives instructions, chooses difficulty, follows a list, selects objects using natural hand movement, receives feedback, and progresses to more challenging tasks.

### DETAILED DESCRIPTION OF THE INVENTION

The following description sets forth illustrative embodiments of the invention in sufficient detail to enable a person skilled in the art to make and use it. These embodiments are provided for the purpose of illustration and are not intended to be limiting. The invention may be embodied in many different forms and should not be construed as limited to the specific examples described herein.

References to “one embodiment” or “an embodiment” mean that a particular feature, structure, or characteristic described in connection with that embodiment is included in at least one embodiment of the invention. The appearances of such phrases in various places are not necessarily all referring to the same embodiment.

Certain features, elements, or steps that are well known to those skilled in the art may not be described in detail so as not to obscure the invention. Likewise, obvious modifications or variations of the described embodiments are considered within the scope of the invention.

Accordingly, the invention provides a system and method for delivering adaptive neuro-rehabilitative therapeutic interventions for individuals with neurocognitive disorders such as Alzheimer's disease, stroke, Parkinson's disease, traumatic brain injury (TBI), and related conditions, as well as individuals experiencing sleep disruption. The system comprises a commercially available virtual reality (VR) or extended reality (XR) headset capable of delivering immersive 360-degree experiences, integrated with synchronized auditory and visual stimulation at a frequency of approximately 40 Hz. This sensory entrainment is designed to support gamma brainwave synchronization and facilitate cognitive and motor engagement during therapeutic sessions.

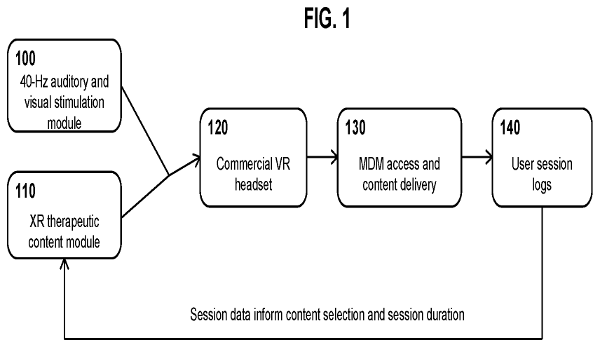

In a preferred embodiment, and with reference to FIG. 1, a VR or XR headset delivers synchronized 40 Hz sensory stimuli while users engage in task-specific therapeutic environments. In preferred embodiments, 40 Hz visual and auditory entrainment is embedded in media content and delivered through the display and speakers of a commercially available headset. Flickering light may be delivered at 480 nm, supporting circadian rhythms and alertness, or at 660 nm, supporting neuroprotection and mitochondrial health. Experiences blended with 480 nm may be used during the day, whereas experiences blended with 660 nm may be used at any time of day. Sound stimulation may use stereo output to deliver 40 Hz pulses or rhythmic auditory stimulation.

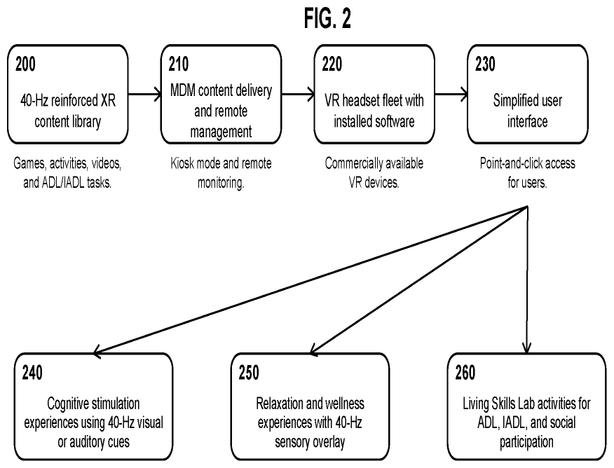

Referring further to FIG. 1 and in combination with FIG. 2, media content may be delivered remotely through MDM or a content delivery management system and may be configured in kiosk mode for simplified access to content. Representative content categories include: (a) cognitive stimulation, including memory games, puzzles, decision-making activities, and reminiscence; (b) relaxation and wellness, including guided meditation, travel, nature immersion, and storytelling; and (c) interactive activities for ADLs, IADLs, foundational skills, and social participation skills.

Interactive environments may simulate everyday tasks such as cooking in a virtual kitchen, grocery shopping with a digital list, dressing using coordinated hand movements, or taking part in social activities or festive environments.

The invention allows for therapeutic customization based on clinical conditions, including Alzheimer's disease, stroke, Parkinson's disease, traumatic brain injury, and sleep disruption. Although the system can record and display performance metrics such as response time or task completion, it does not require machine learning or artificial intelligence.

The XR system may include one or more dashboards for caregivers, clinicians, facility staff, or administrators to monitor session-related information. Such information may include session frequency, session duration, content module selected, task completion, user engagement metrics, tolerance indicators, response accuracy, response time, level progression, and caregiver or clinician notes. The dashboard may display such information over time to support longitudinal progress tracking and to assist caregivers or clinicians in selecting subsequent content modules, session duration, or activity difficulty. In some embodiments, the dashboard supports informed human adjustment of therapeutic content or session parameters without requiring predictive algorithms or machine learning.

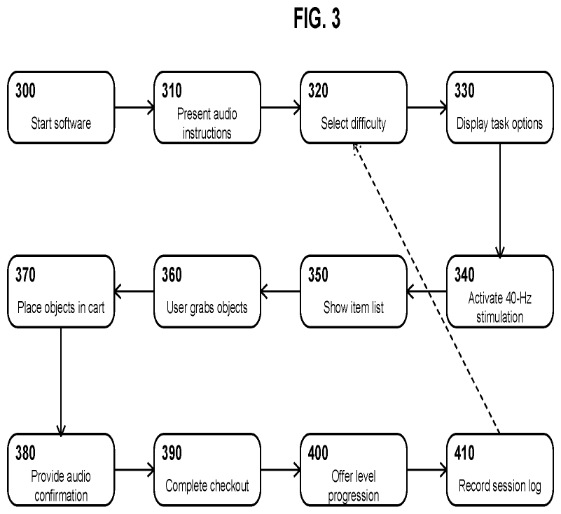

FIG. 3 illustrates a sample use case flow of a shopping simulation. In the example, the user begins at a start page, receives audio instructions, chooses a difficulty level, views a shopping list, selects items using natural hand movements, places selected objects in a cart, receives audio or visual feedback, and may be invited to advance to a more challenging task level. The user may be assisted by caregivers or clinicians during the simulation.

1. A system for delivering therapeutic interventions to individuals with neurocognitive or sleep disorders, the system comprising of one or more virtual reality (VR) or extended reality (XR) headsets configured to render immersive, 360-degree visual environments.

2. The system of claim 4, wherein said visual stimulation is emitted at a wavelength of approximately 480 nm or 660 nm.

3. The system of claim 1, wherein the task-based therapeutic activities comprise bilateral motor tasks requiring coordinated use of both hands.

4. The system of claim 1, wherein the said cognitive games are selected from the group consisting of memory matching tasks, attention tracking, problem-solving challenges, and language-based games.

5. The system of claim 10, further comprising a content management module configured to log session data, including task completion and user engagement metrics.

6. The system of claim 1, wherein the said VR simulations are designed to simulate real-world environments such as kitchens, bathrooms, grocery stores, or public transportation settings.

7. The system of claim 1, wherein the said relaxation modules include guided meditations, nature immersion, music therapy, and calming visual experiences.

8. The system of claim 1, wherein the said VR headset includes stereoscopic visual display and head tracking to enhance immersive engagement.

9. The system of claim 2, wherein the said user interface operates in offline kiosk mode with hand-tracking enabled to simplify session initiation for individuals with cognitive impairments.

10. A method of delivering a therapeutic experience comprising:
initiating a virtual simulation session within a VR or XR environment.

11. The method of claim 15, wherein the collected data is used for generating a personalized therapeutic engagement score for longitudinal progress tracking.

12. The method of claim 12 further comprising simultaneously delivering approximately 40 Hz flickering light and approximately 40 Hz auditory stimulation.

13. The method of claim 13 further comprising guiding a user through functional, cognitive, or relaxation tasks within the virtual simulation session.

14. The method of claim 14 further comprising logging session data for review.

15. The system of claim 3 wherein a 40-Hertz entertainment module is configured to deliver phase-locked visual stimulation and auditory stimulation at a gamma frequency of approximately 40 Hz.

16. The system of claim 5 wherein a media playback system is configured to deliver synchronized visual and auditory stimuli, each modulated at approximately 40 Hz during playback of therapeutic content.

17. The system of claim 6 wherein a user interface is configured to initiate sessions that combine the virtual simulations with phase-locked 40 Hz light and sound stimulation.

18. The system of claim 7, wherein the said VR simulations are designed to simulate real-world Activities of Daily Living (ADL) and Instrumental Activities of Daily Living (IADL) environments with task-based therapeutic activities with bilateral motor tasks requiring coordinated use of both hands.

19. The system of claim 8, wherein there are therapeutic cognitive games.

20. The system of claim 9, wherein there are relaxation modules including guided meditations, nature immersion, music therapy, and calming visual experiences.

1. A system for delivering therapeutic interventions to individuals with neurocognitive or sleep disorders, the system including one or more virtual reality (VR) or extended reality (XR) headsets configured to render immersive, 360-degree visual environments.

2. The system of claim 1, wherein the said VR headset includes stereoscopic visual display and head tracking to enhance immersive engagement.

3. The system of claim 1, wherein the said user interface operates in offline kiosk mode with hand-tracking enabled to simplify session initiation for individuals with cognitive impairments.

4. The system of claim 3 wherein a 40-Hertz entertainment module is configured to deliver phase-locked visual stimulation and auditory stimulation at a gamma frequency of approximately 40 Hz.

5. The system of claim 4, wherein said visual stimulation is emitted at a wavelength of approximately 480 nm or 660 nm.

6. The system of claim 5 wherein a media playback system is configured to deliver synchronized visual and auditory stimuli, each modulated at approximately 40 Hz during playback of therapeutic content.

7. The system of claim 6 wherein a user interface is configured to initiate sessions that combine virtual simulations with phase-locked 40 Hz light and sound stimulation.

8. The system of claim 7, wherein the said VR simulations are designed to simulate real-world Activities of Daily Living (ADL) and Instrumental Activities of Daily Living (IADL) environments with task-based therapeutic activities with bilateral motor tasks requiring coordinated use of both hands.

9. The system of claim 8, wherein there are therapeutic cognitive games.

10. The system of claim 9, wherein there are relaxation modules including guided meditations, nature immersion, music therapy, and calming visual experiences.

11. The system of claim 10, further comprising a content management module configured to log session data, task completion and user engagement metrics.

12. A method of claim 1 delivering a combined therapeutic experience comprising initiating a virtual simulation session within a VR or XR environment.

13. The method of claim 12 further comprising simultaneously delivering approximately 40 Hz flickering light and approximately 40 Hz auditory stimulation.

14. The method of claim 13 further comprising guiding a user through functional, cognitive, or relaxation tasks within the virtual simulation session.

15. The method of claim 14 further comprising logging session data for review.

16. The method of claim 15, wherein the session data is used for generating a personalized therapeutic engagement score for longitudinal progress tracking.
